(19) United States
(12) Patent Application Publication
(10) Pub. No.: US 2015/0107587 A1
(43) Pub. Date: Apr. 23, 2015

(54) ZERO POINT CALIBRATION METHOD AND APPARATUS FOR PRESSURE SENSOR OF ANESTHESIA MACHINE

(71) Applicant: Beijing Aeonmed Co., Ltd., Beijing (CN)

(72) Inventor: Yaodong Zhang, Beijing (CN)

(73) Assignee: BEIJING AEONMED CO., LTD., Beijing (CN)

(21) Appl. No.: 14/396,302

(22) PCT Filed: Oct. 22, 2013
(86) PCT No.: PCT/CN2013/085689

§ 371 Date: Oct. 22, 2014

(30) Foreign Application Priority Data
Dec. 26, 2012 (CN) 201210576231.8

(51) Int. Cl. A61M 16/00; A61M 16/01

(52) U.S. Cl. CPC A61M 16/0003; A61M 16/01; A61M 2016/0027; A61M 2205/70; A61M 2016/0036; A61M 2205/3303; A61M 2230/40; A61M 2205/3344
USPC 128/203.14; 128/203.12

(57) ABSTRACT

Disclosed are a zero point calibration method and apparatus for a pressure sensor of an anesthesia machine. The method comprises: performing zero point calibration on an airway pressure sensor of the anesthesia machine (110); restarting and carrying out startup self-testing (120); and judging whether pressure change values of the airway pressure sensor and an ambient pressure sensor are the same, and if the values are the same, performing zero point calibration on the ambient pressure sensor according to a zero point of the airway pressure sensor (130). By the method of the invention, zero point calibration for a pressure sensor of an anesthesia machine can be realized via a simple operation, thereby avoiding errors in operation.

### TECHNICAL FIELD

The present invention relates to the field of medical appliances, and in particular, to a zero point calibration method and apparatus for a pressure sensor of an anesthesia machine.

### BACKGROUND

In an anesthesia machine, pressure parameters including a airway pressure parameter, an ambient pressure parameter and an air supply pressure parameter are essential. These pressure parameters are all detected by pressure sensors. Generally, a dedicated airway pressure sensor and a dedicated ambient pressure sensor are provided in the anesthesia machine to monitor the airway pressure parameter and the ambient pressure parameter, respectively.

Zero point calibration of the pressure sensors is a vital operation that guarantees the normal use and safety of the anesthesia machine. At present, there mainly exist two zero point calibration modes for a pressure sensor, particularly, in one of the zero point calibration modes, zero point calibration is automatically performed upon the anesthesia is powered on so that the zero point of the pressure sensor is automatically calibrated, and in the other one of the zero point calibration mode, a special zero point calibration operation is performed, that is, a dedicated option or command is set in the anesthesia machine and is used by an operator to place the pressure sensor in a certain working state, and then zero point calibration is performed on the pressure sensor of the anesthesia machine. For the automatic zero point calibration, it is a default that the sensor has a certain pressure curve, but no attention is paid to whether the pressure sensor is actually at a zero pressure state when the anesthesia machine is powered on, thus inaccurate calibration will occur if the pressure sensor in the anesthesia machine powered on is not at the zero pressure state. For the special zero point calibration operation which is complex, an additional standard air pressure gauge is further required to determine the pressure curve, thus it is unfavorable for maintenance of the anesthesia machine in a hospital, and a professional operation is required, thus errors likely occur.

### SUMMARY

Therefore, it is an aspect of the present invention to provide a zero point calibration method and apparatus for a pressure sensor of an anesthesia machine, so that zero point calibration for the pressure sensor of the anesthesia machine may be realized via a simple operation without the need for an additional standard air pressure gauge, and operation errors may be avoided to the maximum extent.

The present invention discloses a zero point calibration method for a pressure sensor of an anesthesia machine, including:
performing zero point calibration on an airway pressure sensor of the anesthesia machine;
restarting the anesthesia machine and carrying out startup self-testing of the anesthesia machine; and
judging whether an air pressure change value detected by the airway pressure sensor and an air pressure change value detected by an ambient pressure sensor are the same, and if the air pressure change values are the same, performing zero point calibration on the ambient pressure sensor according to a zero point of the airway pressure sensor.

Preferably, the method further includes:
prompting to set the airway pressure sensor of the anesthesia machine in a zero pressure state via a prompting apparatus before performing the zero point calibration on the airway pressure sensor of the anesthesia machine.

Preferably, the prompting to set the airway pressure sensor of the anesthesia machine in a zero pressure state includes:
prompting to remove a respiratory pipeline of the anesthesia machine and shut down an electronic flowmeter of the anesthesia machine.

Preferably, the performing zero point calibration on an airway pressure sensor of the anesthesia machine includes:
judging whether the airway pressure sensor is in a zero point qualified range, and if the airway pressure sensor is in the zero point qualified range, performing the zero point calibration on the airway pressure sensor; otherwise, prompting that the airway pressure sensor is invalid.

Preferably, the method further includes:
if the air pressure change values detected by the airway pressure sensor and the ambient pressure sensor are different, prompting that the pressure sensor is invalid.

The present invention further discloses a zero point calibration apparatus for a pressure sensor of an anesthesia machine, including:
a first calibration module, configured for performing zero point calibration on an airway pressure sensor of the anesthesia machine;
a restart module, configured for restarting the anesthesia machine and carrying out startup self-testing of the anesthesia machine; and
a second calibration module, configured for judging whether an air pressure change value detected by the airway pressure sensor and an air pressure change value detected by an ambient pressure sensor are the same, and if the air pressure change values are the same, performing zero point calibration on the ambient pressure sensor according to a zero point of the airway pressure sensor.

Preferably, the apparatus further includes:
a first prompting module, configured for prompting to set the airway pressure sensor of the anesthesia machine in a zero pressure state via a prompting apparatus before performing the zero point calibration on the airway pressure sensor of the anesthesia machine.

Preferably, the prompting to set the airway pressure sensor of the anesthesia machine in a zero pressure state includes:
prompting to remove a respiratory pipeline of the anesthesia machine and shut down an electronic flowmeter of the anesthesia machine.

Preferably, the apparatus further includes a second prompting module, where the first calibration module is configured for judging whether the airway pressure sensor is in a zero point qualified range, and if the airway pressure sensor is in the zero point qualified range, the first calibration module performs the zero point calibration on the airway pressure sensor; otherwise, the second prompting module prompts that the airway pressure sensor is invalid.

Preferably, the apparatus further includes:
a third prompting module, configured for prompting that the pressure sensor is invalid when the air pressure change values detected by the airway pressure sensor and the ambient pressure sensor are different.

In the present disclosure, zero point calibration is performed on merely an airway pressure sensor during the above special zero point calibration, then the anesthesia machine is restarted automatically, and automatic zero point calibration is performed after the restarting, so that situations of the airway pressure sensor and the ambient pressure sensor are judged according to the air pressure change values detected by the airway pressure sensor and the ambient pressure sensor, and zero point calibration is performed on the ambient pressure sensor with reference to the calibrated airway pressure sensor. In this embodiment, the need for a special standard air pressure gauge is eliminated, and the operation flow by an operator is simplified, thus errors in the zero point calibration will not likely occur.

### BRIEF DESCRIPTION OF THE DRAWINGS

FIG. 1 is a flow chart of a zero point calibration method for a pressure sensor of an anesthesia machine according to a first embodiment of the invention;

FIG. 2 is a flow chart of a zero point calibration method for a pressure sensor of an anesthesia machine according to a second embodiment of the invention; and

FIG. 3 is a structural representation of a zero point calibration apparatus for a pressure sensor of an anesthesia machine according to a third embodiment of the invention.

### DETAILED DESCRIPTION OF THE EMBODIMENTS

The technical solutions of the invention will be further illustrated below in conjunction with the accompanying drawings and specific implementations.

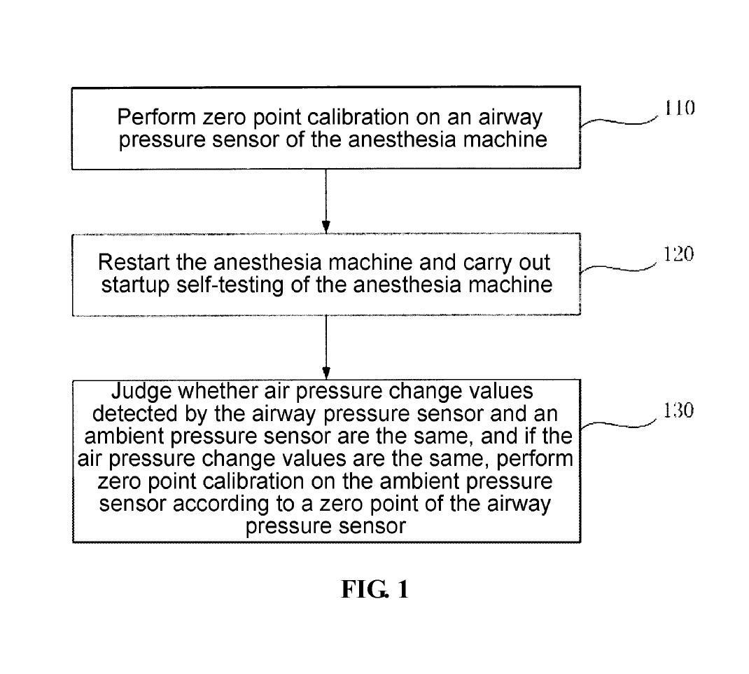

FIG. 1 is a flow chart of a zero point calibration method for a pressure sensor of an anesthesia machine according to the first embodiment of the invention. As shown in FIG. 1, the method includes:

Step 110 of performing zero point calibration on an airway pressure sensor of the anesthesia machine;

Step 120 of restarting the anesthesia machine and carrying out startup self-testing of the anesthesia machine; and

Step 130 of judging whether an air pressure change value detected by the airway pressure sensor and an air pressure change value detected by an ambient pressure sensor are the same, and if the air pressure change values are the same, performing zero point calibration on the ambient pressure sensor according to a zero point of the airway pressure sensor.

Each pressure sensor has a constant pressure characteristic curve, but an error may exist in the zero point value of the pressure sensor, and a certain drift of the zero point value will occur as time and temperature changes. An airway pressure sensor is employed to detect the pressure value of the airway, and is a core part of an anesthesia machine. The accuracy of the airway pressure value is very important for controlling the anesthesia process of the anesthesia machine, thus the airway pressure sensor is required to have high precision, but the measurement range of the airway pressure sensor is small, as a result, the zero point of the airway pressure sensor needs to be calibrated preferentially. Meanwhile, an ambient pressure sensor is employed to detect the ambient pressure parameter, and has a relatively large measurement range. The ambient pressure parameter measured is mainly used for altitude compensation, where the amount of the altitude compensation is a segmented value, thus an error and drift of the zero point may be ignored.

Therefore, in this embodiment, zero point calibration is performed on merely an airway pressure sensor during the above special zero point calibration, then the anesthesia machine is restarted automatically, and automatic zero point calibration is performed after the restarting, so that situations of the airway pressure sensor and the ambient pressure sensor are judged according to the air pressure change values detected by the airway pressure sensor and the ambient pressure sensor, and zero point calibration is performed on the ambient pressure sensor with reference to the calibrated airway pressure sensor. In this embodiment, the need for a special standard air pressure gauge is eliminated, and the operation flow by an operator is simplified, thus errors in the zero point calibration will not likely occur.

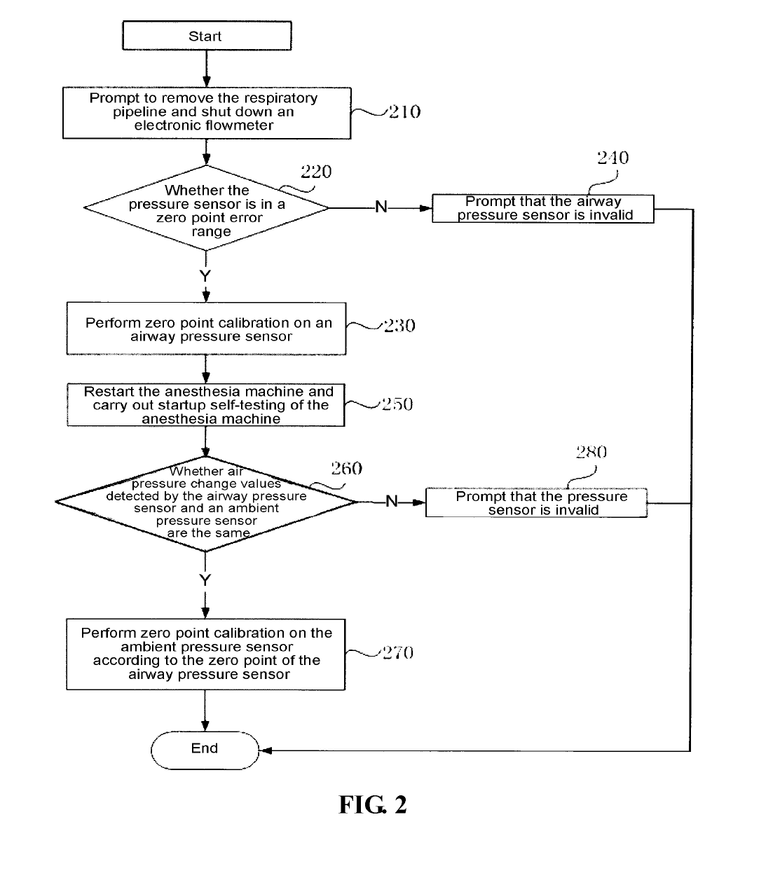

FIG. 2 is a flow chart of a zero point calibration method for a pressure sensor of an anesthesia machine according to the second embodiment of the invention. As shown in FIG. 2, the method includes Steps 210 to 280 below.

Step 210: prompting to set an airway pressure sensor of the anesthesia machine in a zero pressure state via a prompting apparatus.

If the airway pressure sensor is not in the zero pressure state, an error certainly exists in a result of the zero point calibration. Thus, in the method of this embodiment, after an operator chooses to carry out the zero point calibration, the operator is first prompted to remove the respiratory pipeline of the anesthesia machine and shut down an electronic flowmeter, so as to guarantee that the airway pressure sensor is in the zero pressure state.

Step 220: judging whether a zero point of the airway pressure sensor and a zero point of the ambient pressure sensor each are in a qualified range of zero point, and if so, Step 230 is performed; otherwise, Step 240 is performed.

This step is mainly to judge whether a zero point error of the pressure sensor is within a normal range, and the above qualified range of zero point refers to a range of zero point errors that may be corrected; if the zero point error of the pressure sensor is within the normal range, the zero point error may be eliminated via the zero point calibration, but if the zero point error of the pressure sensor exceeds the normal range, the pressure sensor cannot be accurately calibrated even via the zero point calibration.

Step 230: performing the zero point calibration on the airway pressure sensor of the anesthesia machine.

In this step, the zero point calibration is performed on the airway pressure sensor in the zero pressure state in the same way as in the prior art, to eliminate the zero point error caused by the drift of the zero point value.

Step 240: prompting that the airway pressure sensor or the ambient pressure sensor is invalid.

As described above, if the zero point error of the airway pressure sensor or ambient pressure sensor exceeds the normal range, the airway pressure sensor may be invalid and need be replaced or repaired by a professional technician; at this point, a controller of the anesthesia machine prompts the user that the airway pressure sensor is invalid, thus continuous use of the faulted anesthesia machine by the user is avoided.

Step 250: restarting the anesthesia machine, and carrying out startup self-testing of the anesthesia machine.

After the zero point calibration is performed on the airway pressure sensor, the anesthesia machine is restarted, and startup self-testing of the anesthesia machine is carried out. Because the operator has been reminded to place the airway pressure sensor in the zero air pressure state in the previous step, the subsequent process is entirely conducted under the automatic control of the controller, thus during the startup self-testing, the pressure sensor is still in the zero pressure state.

Step 260: judging whether an air pressure change value detected by the airway pressure sensor and an air pressure change value detected by the ambient pressure sensor are the same, and if the air pressure change values are the same, Step 270 is performed; otherwise, Step 280 is performed.

According to the logic of the startup self-testing, the air pressure change value detected by the airway pressure sensor is compared with the air pressure change value detected by the ambient pressure sensor during the startup self-testing, and if the air pressure change values are consistent with each other, it is indicated that the curvatures of the airway pressure sensor and the ambient pressure sensor are correct. Moreover, the current air pressure experienced by the ambient pressure sensor after the airway pressure is excluded, i.e., the atmospheric pressure, or in other words, the zero point value of the ambient pressure sensor, may be calculated according to the calibrated zero point of the airway pressure sensor. If the air pressure change values are inconsistent with each other, it is indicated that one of the above two sensors fails certainly, and the user is prompted about the presence of the invalid pressure sensor.

Step 270: performing zero point calibration on the ambient pressure sensor according to the zero point of the airway pressure sensor.

Specifically, as described above, the zero point drift size of the ambient pressure sensor may be calculated according to the calibrated zero point of the airway pressure sensor, thus the zero point calibration may be performed on the ambient pressure sensor based on the calculated zero point drift size.

Step 280: prompting that the pressure sensor is invalid.

As described above, during the startup self-testing, if it is detected that the air pressure change values detected by the airway pressure sensor and the ambient pressure sensor are different, it is indicated that one of these two sensors fails certainly, and the user needs to be prompted about the presence of the invalid pressure sensor, thus continuous use of the anesthesia machine at the risk of failure by the operator is avoided.

In this embodiment, with the added step of prompting that a pressure sensor is invalid when it is detected that the zero point of the pressure sensor exceeds the zero point error range and it is found during the startup self-testing that the air pressure change values detected by the airway pressure sensor and the ambient pressure sensor are different, a failed pressure sensor may be found in time in the zero point calibration method, and the security of the anesthesia machine may be improved.

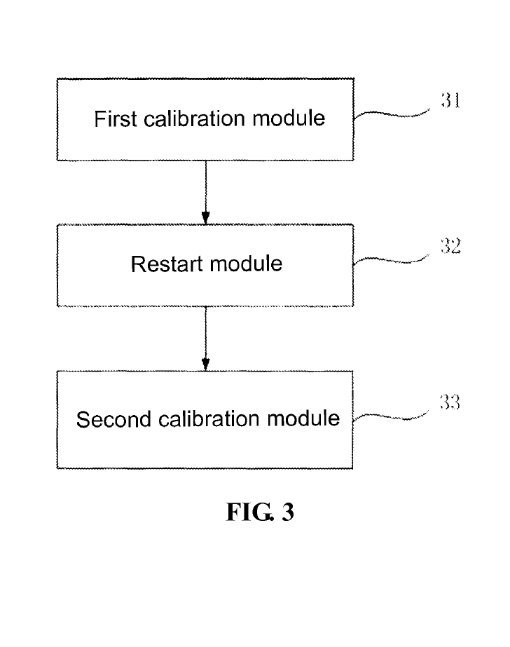

FIG. 3 is a structural representation of a zero point calibration apparatus for a pressure sensor of an anesthesia machine according to the third embodiment of the invention. As shown in FIG. 3, the zero point calibration apparatus 30 for a pressure sensor of an anesthesia machine includes:
a first calibration module 31, configured for performing zero point calibration on an airway pressure sensor of the anesthesia machine;
a restart module 32, configured for restarting the anesthesia machine and carrying out startup self-testing of the anesthesia machine; and
a second calibration module 33, configured for judging whether an air pressure change value detected by the airway pressure sensor and an air pressure change value detected by an ambient pressure sensor are the same, and if the air pressure change values are the same, performing zero point calibration on the ambient pressure sensor according to a zero point of the airway pressure sensor.

In a preferred implementation of this embodiment, the zero point calibration apparatus 30 for a pressure sensor of an anesthesia machine further includes:
a first prompting module, configured for prompting to set the airway pressure sensor of the anesthesia machine in a zero pressure state via a prompting apparatus before performing the zero point calibration on the airway pressure sensor of the anesthesia machine; herein, a user is prompted to remove a respiratory pipeline of the anesthesia machine and shut down an electronic flowmeter of the anesthesia machine, so that the airway pressure sensor of the anesthesia machine is placed in the zero pressure state.

In another preferred implementation of this embodiment, the zero point calibration apparatus 30 for a pressure sensor of an anesthesia machine includes a second prompting module, where the first calibration module is configured for judging whether the airway pressure sensor is in a zero point qualified range, and if the airway pressure sensor is in the zero point qualified range, the first calibration module performs the zero point calibration on the airway pressure sensor; otherwise, the second prompting module prompts that the airway pressure sensor is invalid.

In another preferred implementation of this embodiment, the zero point calibration apparatus 30 for a pressure sensor of an anesthesia machine includes a third prompting module, configured for prompting that the pressure sensor is invalid when air pressure change values detected by the airway pressure sensor and the ambient pressure sensor are different.

Therefore, in this embodiment, zero point calibration is performed on merely an airway pressure sensor during the above special zero point calibration, then the anesthesia machine is restarted automatically, and automatic zero point calibration is performed after the restarting, so that situations of the airway pressure sensor and the ambient pressure sensor are judged according to the air pressure change values detected by the airway pressure sensor and the ambient pressure sensor, and zero point calibration is performed on the ambient pressure sensor with reference to the calibrated airway pressure sensor. In this embodiment, the need for a special standard air pressure gauge is eliminated, and the operation flow by an operator is simplified, thus errors in the zero point calibration will not likely occur.

Apparently, it should be understood by one skilled in the art that, the various modules or steps of the above disclosure may be realized by a general-purpose computing apparatus, and may be centralized on a single computing apparatus or distributed over a network consisted of a plurality of computing apparatuses; optionally, the various modules or steps may be realized by computer-executable software codes, which may be stored on a storage device and executed by a computing apparatus, or the various modules or steps may be embodied by respective integrated circuit modules, or some of the various modules or steps may be embodied by a single integrated circuit module. Thus, the invention is not limited to any combination of certain hardware or software.

The above description only shows some preferred embodiments of the invention, rather than limiting the scope of the invention. Various variations and modifications can be made on the invention by one skilled in the art. Therefore, all the modifications, equivalent substitutions and improvements made within the spirit and principle of the invention should fall within the protection scope of the invention.

1. A zero point calibration method for a pressure sensor of an anesthesia machine, comprising:
performing zero point calibration on an airway pressure sensor of the anesthesia machine;
restarting the anesthesia machine and carrying out startup self-testing of the anesthesia machine; and
judging whether an air pressure change value detected by the airway pressure sensor and an air pressure change value detected by an ambient pressure sensor are the same, and if the air pressure change values are the same, performing zero point calibration on the ambient pressure sensor according to a zero point of the airway pressure sensor.

2. The zero point calibration method for a pressure sensor of an anesthesia machine according to claim 1, comprising:
prompting to set the airway pressure sensor of the anesthesia machine in a zero pressure state via a prompting apparatus before performing the zero point calibration on the airway pressure sensor of the anesthesia machine.

3. The zero point calibration method for a pressure sensor of an anesthesia machine according to claim 2, wherein the prompting to set the airway pressure sensor of the anesthesia machine in a zero pressure state comprises:
prompting to remove a respiratory pipeline of the anesthesia machine and shut down an electronic flowmeter of the anesthesia machine.

4. The zero point calibration method for a pressure sensor of an anesthesia machine according to claim 1, wherein the performing zero point calibration on an airway pressure sensor of the anesthesia machine comprises:
judging whether the airway pressure sensor is in a zero point qualified range, and if the airway pressure sensor is in the zero point qualified range, performing the zero point calibration on the airway pressure sensor; otherwise, prompting that the airway pressure sensor is invalid.

5. The zero point calibration method for a pressure sensor of an anesthesia machine according to claim 1, further comprising:
if the air pressure change values detected by the airway pressure sensor and the ambient pressure sensor are different, prompting that one or more of the airway pressure sensor and the ambient pressure sensor are invalid.

6. A zero point calibration apparatus for a pressure sensor of an anesthesia machine, comprising:
a first calibration module, configured for performing zero point calibration on an airway pressure sensor of the anesthesia machine;
a restart module, configured for restarting the anesthesia machine and carrying out startup self-testing of the anesthesia machine; and
a second calibration module, configured for judging whether an air pressure change value detected by the airway pressure sensor and an air pressure change value detected by an ambient pressure sensor are the same, and if the air pressure change values are the same, performing zero point calibration on the ambient pressure sensor according to a zero point of the airway pressure sensor.

7. The zero point calibration apparatus for a pressure sensor of an anesthesia machine according to claim 6, further comprising:
a first prompting module, configured for prompting to set the airway pressure sensor of the anesthesia machine in a zero pressure state via a prompting apparatus before performing the zero point calibration on the airway pressure sensor of the anesthesia machine.

8. The zero point calibration apparatus for a pressure sensor of an anesthesia machine according to claim 7, wherein the prompting to set the airway pressure sensor of the anesthesia machine in a zero pressure state comprises:
prompting to remove a respiratory pipeline of the anesthesia machine and shut down an electronic flowmeter of the anesthesia machine.

9. The zero point calibration apparatus for a pressure sensor of an anesthesia machine according to claim 6, further comprising a second prompting module, wherein the first calibration module is configured for judging whether the airway pressure sensor is in a zero point qualified range, and if the airway pressure sensor is in the zero point qualified range, the first calibration module performs the zero point calibration on the airway pressure sensor; otherwise, the second prompting module prompts that the airway pressure sensor is invalid.

10. The zero point calibration method for a pressure sensor of an anesthesia machine according to claim 6, further comprising:
a third prompting module, configured for prompting that one or more of the airway pressure sensor and the ambient pressure sensor are invalid when the air pressure change values detected by the airway pressure sensor and the ambient pressure sensor are different.
